(19) United States
(12) Patent Application Publication
(10) Pub. No.: US 2015/0238943 A1
(43) Pub. Date: Aug. 27, 2015

(54) CATALYST COMPRISING SILVER OXIDE AND CALCIUM CARBONATE, METHOD FOR THE MANUFACTURE OF THE CATALYST AND ITS USE FOR THE DESTRUCTION OF STERILANT

(71) Applicant: JOHNSON MATTHEY PUBLIC LIMITED COMPANY, London (GB)

(72) Inventor: Martyn Vincent Twigg, Cambridgeshire (GB)

(21) Appl. No.: 14/429,805

(22) PCT Filed: Sep. 12, 2013
(86) PCT No.: PCT/GB2013/052383

§ 371 Date: Mar. 20, 2015

(30) Foreign Application Priority Data
Sep. 21, 2012 (GB) 1216857.1

(51) Int. Cl. B01J 27/232; B01J 37/02; B01J 37/04; B01J 35/04; B01J 19/24; B01J 21/06; B01J 23/68; B01J 35/00; B01J 23/50; A61L 9/04

(52) U.S. Cl. CPC B01J 27/232; B01J 23/50; B01J 37/0215; B01J 37/04; A61L 9/04; B01J 19/24; B01J 21/063; B01J 23/688; B01J 35/0006; B01J 35/04; B01J 2219/24

(57) ABSTRACT

Disclosed are catalysts, and in particular catalysts including silver oxide and calcium carbonate. The catalysts may be used in the destruction of sterilants such as ozone and hydrogen peroxide. The present inventors have found that including calcium carbonate in catalysts for the destruction of sterilant substances such as ozone and peroxide enhances the performance of the catalyst and increases its lifetime. The beneficial effect of including calcium carbonate is particularly observed for catalysts including silver oxide, such as those including silver oxide and titanium dioxide.

### FIELD OF THE INVENTION

The present invention relates to catalysts, and in particular to catalysts comprising silver oxide and calcium carbonate. The catalysts may be used in the destruction of sterilants such as ozone and hydrogen peroxide.

### BACKGROUND OF THE INVENTION

It is known to use sterilants such as ozone and hydrogen peroxide for removing bacteria, other pathogenic microorganisms and other contaminants from an enclosed area environment, such as areas in hospitals. However, such sterilants typically are also toxic or hazardous to humans and animals. Accordingly, care has to be taken to reduce the concentration of the sterilant in the environment to safe levels before allowing access after sterilisation.

It is known that it may be advantageous to humidify the atmosphere before or during sterilisation. For example, the atmosphere may be humidified by spraying water droplets into the environment to be sterilised, or by passing steam into the environment to be sterilised. High levels of humidity are considered to be particularly advantageous. For example, a relative humidity of about 70% or more may be preferred.

Humidity is particularly desirable where ozone is supplied as the sterilant. This is because the presence of water seems to enhance the effectiveness of ozone as a sterilant, perhaps through the reaction of ozone to form hydroxyl radicals, which are particularly powerful oxidants. Furthermore, these hydroxyl radicals may combine to form hydrogen peroxide, which has powerful antiseptic properties.

In order to return the environment to a safe state after sterilisation, the sterilant is typically contacted with a catalyst which promotes its decomposition. Known catalysts for the decomposition e.g. of ozone include formulations containing metal components such as platinum, or oxides of manganese and other transition metal elements. Surprisingly, the present inventors have found that platinum catalysts do not work well in sterilisation applications, where high ozone levels and high humidity levels are employed at ambient temperatures. Moderately good initial performance was observed, but deactivation was rapid. This may be due to strong adsorption of water and/or oxygen species on the active sites. Highly-loaded MnO₂ catalysts had good initial activity, but while they were initially better than platinum they deactivated quickly in use. The present inventors have found that removal of adsorbed species by vacuum oven treatment at 150° C. (e.g. overnight) restored a portion of the lost activity, but the combination of rapid deactivation and slow regeneration means that this catalyst is not a practical solution.

It has also been proposed to use silver oxide/MnO₂ catalysts. However, the present inventors have found that these catalysts also deactivate quickly under the conditions of high humidity and high ozone concentrations at low temperature. The humidity in particular may be responsible for the observed deactivation.

WO2010/067120 describes improved catalysts for the destruction of ozone or other sterilant, which have good performance in the sterilisation conditions discussed above. The catalysts of WO2010/067120 comprise titanium dioxide, silver oxide, and optionally MnO₂ and silica. However, there remains a need for improved catalysts, particularly with enhanced catalytic activity and/or increased lifetime.

### SUMMARY OF THE INVENTION

The present inventors have found that including calcium carbonate in catalysts for the destruction of sterilant substances such as ozone and peroxide may enhance the performance of the catalyst and/or increase its lifetime. The beneficial effect of including calcium carbonate is particularly observed for catalysts comprising silver oxide, such as those comprising silver oxide and titanium dioxide.

Accordingly, in a first preferred aspect the present invention provides a catalyst for the destruction of sterilant, the catalyst comprising silver oxide and calcium carbonate. Preferably, the catalyst further comprises titanium dioxide.

The catalyst may be supported on a substrate. Accordingly, in a second preferred aspect the present invention provides a catalyst body comprising the catalyst of the invention supported on a substrate. The substrate may be, for example, a ceramic or metal substrate, such as a ceramic or metal honeycomb substrate. The catalyst may be coated onto the substrate, e.g. by wash coating.

In a third preferred aspect, the present invention provides a method for the manufacture of a catalyst according to the first aspect, comprising combining silver oxide, calcium carbonate and optionally further components to form the catalyst. For example, titanium dioxide may additionally be combined with the silver oxide and calcium carbonate. The method may further comprise depositing the catalyst on a substrate, e.g. by wash coating.

In a further preferred aspect, the present invention provides use of a catalyst of the present invention for the destruction of sterilant.

In a further preferred aspect, the present invention provides a method for sterilising an enclosed space, comprising supplying sterilant to the atmosphere in the space to sterilise the space, and subsequently catalytically destroying the sterilant by contacting the atmosphere with a catalyst of the present invention. For example, the atmosphere may be passed over the catalyst. The method preferably further comprises humidifying the atmosphere in the space before or during sterilisation.

The sterilant in the atmosphere may be catalytically destroyed using sterilant gas removal apparatus. Accordingly, in a further preferred aspect the present invention provides sterilant gas removal apparatus comprising a flow path along which gas (e.g. the atmosphere from which the sterilant is to be removed) may flow, the flow path having catalyst of the present invention provided therein. Preferably the apparatus is portable, so that it can be moved into the environment to be sterilised (e.g. prior to introduction of the sterilant) and subsequently operated to remove sterilant from the atmosphere in the sterilised environment.

### DETAILED DESCRIPTION OF THE INVENTION

Further preferred or optional features of the invention will now be set out. Any aspect of the invention may be combined with any other aspect, unless the context demands otherwise. Any of the preferred or optional features of an aspect may be combined, singly or in combination, with any aspect of the invention, unless the context demands otherwise.

The catalyst of the present invention comprises silver oxide and calcium carbonate. Preferably, the catalyst further comprises titanium dioxide. It may optionally further comprise one or more additional components. For example, the catalyst may optionally further comprise MnO₂ and/or the catalyst may optionally further comprise silica.

Typically, the catalyst contains at least 50 wt % of silver oxide. Preferably, it contains at least 60 wt % or at least 70 wt % of silver oxide. It may contain 90 wt % or less, 85 wt % or less or 80 wt % or less of silver oxide. However, where the catalyst comprises MnO₂, typically the MnO₂ replaces some of the silver oxide. Accordingly, where the catalyst comprises MnO₂, the catalyst typically contains at least 50 wt % of MnO₂ and silver oxide taken together, preferably at least 60 wt % or at least 70 wt % of MnO₂ and silver oxide taken together. In this case, typically the catalyst includes at least 30 wt %, at least 40 wt % or at least 50 wt % of silver oxide. The catalyst may contain 90 wt % or less, 85 wt % or less or 80 wt % or less of MnO₂ and silver oxide taken together.

Preferably, the catalyst contains at least 2 wt % of calcium carbonate. More preferably, the catalyst contains at least 3 wt %, at least 4 wt %, at least 5 wt %, at least 6 wt %, at least 7 wt %, at least 8 wt %, at least 9 wt %, or at least 10 wt % of calcium carbonate. The catalyst may comprise 30 wt % or less of calcium carbonate, more preferably 25 wt % or less, 20 wt % or less or 15 wt % or less of calcium carbonate.

Preferably, the catalyst contains at least 5 wt % of titanium dioxide. More preferably, the catalyst contains at least 6 wt %, at least 7 wt %, at least 8 wt %, at least 9 wt %, or at least 10 wt % of titanium dioxide. The catalyst may comprise 30 wt % or less of titanium dioxide, more preferably 25 wt % or less, 20 wt % or less or 15 wt % or less of titanium.

Typically, the catalyst comprises a small amount of residual thickener, which may be employed in the wash-coating process. The catalyst typically includes less than 5 wt % of residual thickener, for example less than 4, 3, 2, or 1.5 wt % of residual thickener. The thickener may be, for example, xanthan gum.

The catalyst may further comprise silica. The presence of silica as a binder in a wash coat is generally expected to improve wash coat adhesion. For example, the catalyst may comprise up to 20 wt % silica, preferably up to 15 wt %, up to 10 wt % or up to 5 wt % silica.

The catalyst is typically supported on a substrate. Typically, the substrate is honeycomb substrate having a plurality of parallel channels, and may be made from a suitable ceramic or metal (e.g. stainless steel) material.

The honeycomb substrate may be of the “flow-though” type, in which channels of the honeycomb are open at each end. In this way, the atmosphere to be treated by the catalyst may be flowed along the channels of the honeycomb, and as a result may be brought into contact with catalyst deposited on the walls of the channels. This arrangement has the advantage of having a low pressure drop. Low pressure drop is important because of fan size limitations, especially in portable equipment.

Alternatively, the honeycomb support may be a “wall-flow” filter, in which alternate channel ends are blocked so that the atmosphere to be treated is forced to flow through the channel walls. The catalyst may be deposited on the surface of the walls, and within the walls (e.g. within pores in the walls). Therefore, this arrangement may allow the catalyst to be used more effectively, at the expense of increased backpressure.

Where the support is a honeycomb, typically the catalyst and support is housed in a metal (e.g. stainless steel) cylinder retained with a ceramic mat material, and having suitable coupling devices at its ends. Such arrangements are well known for vehicle catalytic converters.

Foamed or pellet catalyst/support types can also be used. Typically, these are held within a suitable container, preferably made from stainless steel or other ozone resistant material. Alternative supports such as high surface area sintered metal monoliths, static mixers and partial filter constructions may be used.

The catalyst may be coated onto the substrate e.g. by wash-coating. The skilled person will be aware of suitable methods conventionally employed in wash-coating catalytically active components onto supports.

In a typical method for the manufacture of manufacture of the catalyst, the catalyst components are combined and then milled, e.g. using a ball mill to provide the desired particle size. Where the catalyst is to be wash coated onto a substrate, the milled catalyst is usually formed into a slurry, e.g. in water. Thickener (such as xanthan gum) may be added to provide a wash coat slurry having the desired viscosity. A suitable dosage of wash coat onto the surface of the catalyst body may be about 6.5 g/in³.

As discussed above, the catalysts of the present invention are suitable for the destruction of sterilant, such as sterilant gas. Typically, the sterilant comprised ozone, and in the methods of the present invention, typically the sterilant is supplied as a gas comprising ozone. However, as explained above, during the sterilisation process, ozone supplied as sterilant may undergo chemical reaction to produce other sterilant substances. Accordingly, the sterilant destroyed by the catalyst may comprise, alternatively or in addition to ozone itself, sterilant derived from ozone. Sterilants which may be derived from ozone include hydroxyl radicals and/or peroxide (e.g. hydrogen peroxide).

The present invention provides methods for sterilising an enclosed space. The nature of the enclosed space to be sterilised is not particularly limited. For example, it may include areas where plants are grown (e.g. greenhouses); food processing areas (e.g. a kitchen or factory producing food products); hotel rooms; conference centres; areas in hospitals (including for example isolation areas for isolation of infectious disease and/or for immune-compromised patients) and other medical facilities including clinics and ambulances; dwellings; and places were animals are kept (especially quarantine areas).

The contaminants to be removed by sterilisation are not particularly limited in the present invention. For example, the contaminants may be of biological or synthetic origin, and may include bacterial, viral and other pathogens, as well as toxic agents (e.g. synthetic toxic agents).

As mentioned above, the method of the present invention preferably comprises humidifying the atmosphere in the space before or during sterilisation. For example, the atmosphere may be humidified by spraying water droplets into the environment to be sterilised, or by passing steam into the environment to be sterilised. High levels of humidity are considered to be particularly advantageous. For example, a relative humidity of about 70% or more may be preferred.

Ozone may be produced from a suitable ozone generator, such as a generator which acts by irradiating oxygen with ultraviolet radiation, or electrical techniques such as those involving corona discharge or plasma formation. Preferably the source of oxygen contains a only a small amount of nitrogen (e.g. less than 15%) to minimise the formation of undesirable nitrogen oxides. Accordingly, while air may be used as the source of ozone, it is preferable to use pure oxygen or oxygen-enriched air.

During sterilisation, the atmosphere to be sterilised should contain a sufficient concentration of sterilant to provide efficient sterilisation. Similarly, the concentration of sterilant should be maintained for a time sufficient to provide the necessary sterilisation. Of course, the suitable time and concentration will depend on a number of factors, including for example the degree of contamination. For example, sterilant (e.g. ozone) may be supplied to the enclosed space to be sterilised to provide at least 10 ppm sterilant concentration, more preferably at least 20 ppm. Suitable upper limits for zone concentration are 50 ppm, or 40 ppm. Preferably, the concentration of sterilant is maintained for at least about 10 minutes, at least about 20 minutes or at least about 30 minutes. Suitable upper limits for maintaining the concentration of sterilant are 120 minutes or 60 minutes.

After sterilisation, the atmosphere from within the environment is contacted with a catalyst of the present invention to catalytically destroy the sterilant. Preferably, the atmosphere is flowed over the catalyst. For example, the atmosphere may be circulated over the catalyst, e.g. using a fan. The atmosphere may be filtered to remove particulate matter, before, during or after contact with the catalyst. It may be desirable to filter the atmosphere before prior to contact with the catalyst to avoid contamination of the catalyst. However, where the catalyst is deposited on a filter-type support, it will be understood that filtration may be simultaneous with catalyst contact.

Silver containing catalysts can be susceptible to poisoning, especially at low temperature, and particularly by sulphur compounds in either low or high oxidation state e.g. H₂S and SO₂. In use, the atmosphere to be treated is passed over the catalyst. The means that the catalyst contacts a large volume of air, and as a result small amounts of poison can rapidly cause at least some deactivation. Therefore, it may be preferable to protect the catalyst by providing an upstream guard material that has a high affinity for poisons. For example the guard material may comprise, for example, high surface area zinc oxide for trapping H₂S, and/or an alkalised high surface area material, such as alkalised alumina, to capture halides. Different guard materials may be provided in discreet layers, or mixed together. Guard materials may be in the form of pellets or other solid form in a suitable container, or alternatively may be coated onto a flow-through ceramic or metal monolithic honeycomb.

Conveniently, the sterilisation and sterilant destruction processes are carried out at ambient temperatures and pressures. However, higher temperatures may be involved, e.g. of about 200° C., to reduce deactivation of the catalyst, but typically this is inconvenient in the sterilisation applications envisaged.

The catalyst may readily be regenerated by heat treatment in air at moderate temperatures. For example, heating in air at 150° C. typically converts all or substantially all of the higher oxidation state material into the active Ag′ oxide form. The catalyst may be regenerated in situ or in a dedicated apparatus. A heater may be provided, e.g. upstream of the catalyst may be provided to increase the temperature of the air for catalyst regeneration. Typically, it is preferable to simultaneously reduce the flow rate of air over the catalyst. Suitable temperatures for regeneration are in the range 130-250° C., for a period of from 5 minutes to 10 hours, conveniently for 15 minutes to 5 hours. An advantageous regeneration regime could be to regenerate the catalyst relatively frequently for short periods, so that the catalyst maintains its performance over extended periods of time.

The sterilisation method may be controlled by a computer system. For example, the computer system may control the humidification, the supply of sterilant to maintain the desired sterilant levels, and the flowing of the atmosphere over the catalyst to destroy the sterilant. The system may, for example, be provided with sensors and feedback systems to provide this control. Furthermore, predictive computer models can be used to estimate the time needed for sterilisation, and the time needed for sterilant destruction after sterilisation.

The present invention provides sterilant gas removal apparatus. The apparatus comprises a flow path along which gas (e.g. the atmosphere from which the sterilant is to be removed) may flow, the flow path having catalyst (or catalyst body) of the present invention provided therein. Preferably the apparatus is portable, so that it can be moved into the environment to be sterilised (e.g. prior to introduction of the sterilant) and subsequently operated to remove sterilant from the atmosphere in the sterilised environment.

The apparatus may be powered by mains supply or by battery power (e.g. internal rechargeable batteries which are periodically recharged). The apparatus may comprise filter for removing particulate matter from the atmosphere in the environment to be sterilised. It may comprise guard material located upstream of the catalyst to protect the catalyst from poisons as described above. It may comprise a computer system for controlling the sterilisation and/or sterilant removal processes as described above.

Multiple flow paths may be provided, each having catalyst provided therein. In this way, multiple regions of catalyst (or catalyst body) can be employed in parallel. Each flow path may be provided with its own fan for effecting flow of the atmosphere over the catalyst. This can provide additional flexibility to the process.

### Examples

### Preparation of a Catalyst (Ag₂O/TiO₂/CaCO₃)

To deionised water (1.2 litre) was added with stirring with a high shear mixer, silver oxide (Johnson Matthey) (1000 g), TiO₂ (DT-51, Cristal Global) (135 g), and CaCO₃ (150 g) to give a well formed slurry. This was then ball milled using ceria/zirconia balls for 3 hours to give a d₅₀ particle size of less than 5 microns. Deionised water was then added and a xanthan gum (Rhodopol, Rhone-Poulenc SA) (20.2 g) was added with stirring to give a coating mixture with properties enabling easy application to a cordierite honeycomb 10.5 inched diameter 6 inches high having 400 square channels per square inch with wall thickness of 6/1000 inch. Excess wash coat was removed by high pressure gun, and after drying in a flow of air at 90° C. for 1 hour the resulting catalyst body had 1254 g of wash coat.

### Catalyst Performance

Catalysts were tested for their ability to destroy ozone at room temperature and pressure. The catalysts were prepared according to the procedure given above. The following catalyst compositions were tested:

| | MnO₂/g | Ag₂O/g | TiO₂/g | CaCO₃/g | Silica/g |
|---|---|---|---|---|---|
| Example 1 | — | 1000 | 135 | 65 | — |
| Example 2 | — | 1000 | 135 | 150 | — |
| Example 3 | — | 1000 | 135 | 300 | — |
| Comparative | — | 986 | 260.5 | — | — |
| Example 1 | | | | | |
| Comparative | 441.5 | 441.5 | 155.8 | — | 260 |
| Example 2 | | | | | (40% solution) |

The results are given in the table below:

| | | Ozone Half Life/ | Ozone |
|---|---|---|---|
| | Average Humidity | min | Removed |
| Example 1 | 85% | 3.77 | 97% |
| Example 2 | 79% | 4.14 | 97% |
| Example 3 - run 1 | 80% | 1.46 | Not |
| | | | measured |
| Example 3 - run 2 | Not | 1.50 | 81.9% |
| | measured | | |
| Comparative | 85% | 4.14 | 77% |
| Example 1 | | | |
| Comparative | 88% | 4.90 | 62% |
| Example 2 | | | |

The results demonstrate that including calcium carbonate in the catalyst composition improves catalyst performance. In particular, a lower half life for ozone can be achieved, and a higher percentage of ozone removed.

The catalyst performance testing reported here was carried out by Steritrox Limited.

1. A catalyst for the destruction of sterilant, the catalyst comprising silver oxide and calcium carbonate.

2. A catalyst according to claim 1, further comprising titanium dioxide.

3. A catalyst according to claim 1, wherein the catalyst contains at least 50 wt % of silver oxide.

4. A catalyst according to claim 1, wherein the catalyst comprises MnO₂, and wherein the catalyst contains at least 60 wt % of MnO₂ and silver oxide taken together.

5. A catalyst according to claim 1, wherein the catalyst contains at least 5 wt % of calcium carbonate.

6. A catalyst according to claim 1, wherein the catalyst contains at least 5 wt % of titanium dioxide.

7. A catalyst according to claim 1 wherein the catalyst contains less than 5 wt % of residual thickener such as xanthan gum.

8. A catalyst body comprising the catalyst as defined in claim 1 supported on a substrate.

9. A catalyst body according to claim 8 wherein the substrate is a honeycomb substrate.

10. A method for the manufacture of a catalyst as defined in claim 1, comprising combining silver oxide, calcium carbonate and optionally further components to form the catalyst.

11. A method according to claim 10, further comprising depositing the catalyst on a substrate, e.g. by wash coating.

12. A method for destruction of sterilant, comprising applying an effective amount of the catalyst of claim 1.

13. A method for sterilising an enclosed space, comprising supplying sterilant to the atmosphere in the space to sterilise the space, and subsequently catalytically destroying the sterilant by contacting the atmosphere with a catalyst as defined in claim 1.

14. Sterilant gas removal apparatus comprising a flow path along which gas comprising sterilant may flow, the flow path having catalyst as defined in claim 1 provided therein.

15. A catalyst according to claim 2, wherein the catalyst contains at least 50 wt % of silver oxide.

16. A catalyst according to claim 2, wherein the catalyst comprises MnO₂, and wherein the catalyst contains at least 60 wt % of MnO₂ and silver oxide taken together.

17. A catalyst according to claim 3, wherein the catalyst comprises MnO₂, and wherein the catalyst contains at least 60 wt % of MnO₂ and silver oxide taken together.

18. A catalyst according to claim 2, wherein the catalyst contains at least 5 wt % of calcium carbonate.

19. A catalyst according to claim 3, wherein the catalyst contains at least 5 wt % of calcium carbonate.

20. A catalyst according to claim 4, wherein the catalyst contains at least 5 wt % of calcium carbonate.
